(19) United States
(12) Patent Application Publication
(10) Pub. No.: US 2014/0296984 A1
(43) Pub. Date: Oct. 2, 2014

(54) CAGE SYSTEM

(71) Applicant: Mohammad ETMINAN, Houston, TX (US)

(72) Inventor: Mohammad ETMINAN, Houston, TX (US)

(21) Appl. No.: 14/242,255

(22) Filed: Apr. 1, 2014

Related U.S. Application Data
(60) Provisional application No. 61/807,012, filed on Apr. 1, 2013.

(51) Int. Cl. A61F 2/44

(52) U.S. Cl. CPC A61F 2/442
USPC 623/17.16

(57) ABSTRACT

A system for intervetebral fusion that includes a base member with a fist support and a second support, and an end member configured to be assembled with the base member to define a space for reception of graft material, each support member including a first sidewall, a second sidewall spaced from the first sidewall and an end wall connecting the two sidewalls.

### CROSS REFERENCE TO RELATED APPLICATIONS

The present application claims priority to U.S. Provisional Patent Application No. 61/807,012, filed Apr. 1, 2013, the entire contents of which are incorporated by reference herein.

### FIELD OF INVENTION

Disclosed below is a novel concept for intervertebral cage designs.

### BACKGROUND

Intervertebral cages are well known. One known configuration is comprised of an endless, contiguous wall defining an interior space that is accessible from two opposite, open sides.

Another known configuration includes two pieces that can be integrated to define a cage. An example of such a configuration is disclosed in Falahee, U.S. Patent Publication No. 2009/0030519.

In U.S. Pat. No. 8,491,658 (incorporated by reference), the inventor of the present application has proposed a two-piece cage design that includes a base member having three sidewalls and an end member that is coupled to the base member to define a cage. Disclosed in U.S. Pat. No. 8,491,658, a two-piece cage can include a generally elongated U-shaped base member having two opposing sidewalls each with a terminal, free end and an end member that mates and preferably locks with the base member to define a cage. The base member and the end member in a two-piece cage system include mating parts that register with one another so that the two pieces may be assembled in-situ; i.e. during the operation.

A two-piece intervertebral cage can be made from a single material (e.g. a metal) or a composite material. The material selected for the cage will usually have a modulus of elasticity close to the modulus of elasticity of human bone and thus, relatively speaking, may be rather compliant.

To insert a cage between adjacent vertebrae, a space is defined by removing all the disc residing between the adjacent vertebrae and possibly a portion of the vertebrae. The space so defined includes two opposite sides each side extending between the adjacent vertebrae. Generally speaking, after disc removal, one side of the defined space is higher than the other side. Thus, to ensure a stable and an intimate contact between the cage and the adjacent vertebrae a surgeon needs to insert (often with great difficulty) a much larger cage into the defined space to ensure that contact between the sidewalls of the cage and the adjacent vertebrae are intimate and stable.

### SUMMARY OF THE INVENTION

A cage system according to the present invention includes a base member assembly and an end member that can be integrated (i.e. coupled to) with the base member assembly to realize an intervertebral cage.

According to one aspect of the present invention, the base member assembly includes two supports, and at least one link that couples the supports to one another. According to an aspect of the present invention, the link sets the height of one side of the cage.

In the first embodiment, the two supports are hingedly coupled along one side thereof and the link couples the other, opposing sides of the two supports.

In the second embodiment, the two supports are independent bodies and are coupled to one another with two links, each link coupling a side of one support to a respective side of the other support.

A system for intervetebral fusion according to the present invention includes an intervetebral cage having a base member and an end member. The base member includes a first support and a second support, the first support and the second support each having a first sidewall, a second sidewall spaced from the first sidewall, and an end wall connecting the first sidewall to the second sidewall. The end member is configured for assembly with the base member to define a space to receive bone graft material.

The sidewalls in each support may be generally parallel to one another. The end member may be configured for assembly to the ends of the sidewalls of the first and the second support members.

In the first embodiment, the first sidewalls of the first and the second support members are hingedly coupled to one another, and the second sidewalls may be displaced (rotate) relative to one another. A link may be provided to couple the second sidewalls of the first and the second support members. The link may be used to set the spacing between the second sidewalls.

In the second embodiment, the supports are not hingedly coupled to rotate relative to one another. In the second embodiment, the first and the second supports may be coupled to one another with two links.

A link in a system according to the present invention may include a wedge or wedges that are received between the sidewalls to set the spacing. The link may further include lobes each received in a respective groove defined in a respective sidewalls. The link may further include anchors each received in a respective receptacle defined in a respective sidewall.

The cage may further include a first tie and a second tie. The end member may be configured to receive each tie so that it may be assembled with the base member. Thus, for example, the end member may include an opening therein each for receiving a respective tie. In the first embodiment, the first tie may couple the first and the second support, and the first and the second support may rotate about the first tie. In the first embodiment, the link may include the second tie. In the second embodiment, one link may include the first tie and the second link may include the second tie.

A system according to the second embodiment of the present invention may be provided with a dilator to set the spacing between the first and the second support members.

The end member in a system according to the present invention may include a plurality of apertures each for receiving a respective screw to further secure the end plate to a vertebrae.

A system according to the present invention may further include a containment plate receivable inside the base member to close a gap between the first and the second supports to assist the retention of graft material.

Other features and advantages of the present invention will become apparent from the following description of the invention which refers to the accompanying drawings.

### BRIEF DESCRIPTION OF THE FIGURES

FIG. 1 depicts a top plan view of a base member assembly of a multi-piece cage system according to a first embodiment of the present invention.

FIG. 2A depicts a top plan view of an end member of a multi-piece cage system according to the present invention.

FIG. 2B depicts a side plan view of the end member depicted in FIG. 2A, viewed in the direction of arrow 2B.

FIG. 3 depicts an end member and a base member assembly of a multi-piece cage system according to the first embodiment of the present invention in a partially assembled state.

FIG. 4 depicts an end member and a base member assembly of a multi-piece cage system according to the first embodiment of the present invention in an assembled state.

FIG. 5 is a top plan view of two hinged supports (in an open state) used in a base member assembly of a cage according to the first embodiment of the present invention.

FIG. 6A is a view of the hinged supports shown in FIG. 5 in a closed state, viewed in the direction of arrow 6A in FIG. 5.

FIG. 6B is a side plan the hinged supports shown in FIG. 5 in a closed state, viewed in the direction of arrow 6B in FIG. 5.

FIG. 6C is a side plan view of the hinged supports shown in FIG. 5 in a closed state, viewed in the direction of arrow 6C in FIG. 5.

FIG. 7 is a cross-sectional view taken along line 7-7, viewed in the direction of the arrows.

FIG. 8 is a side plan view of a link used in a base member assembly according to the present invention.

FIG. 9 is a cross-sectional view taken along line 9-9 in FIG. 8, viewed in the direction of the arrows.

FIG. 10 shows a view of a base member assembly (in the direction of arrows 10-10 in FIG. 1) installed between two adjacent vertebrae.

FIG. 11 is a cross-sectional view along line 11-11 in FIG. 1, viewed in the direction of the arrows.

FIG. 12 is a cross-sectional view along line 12-12 in FIG. 1, viewed in the direction of the arrows.

FIG. 13 depicts a top plan view of a base member assembly of a multi-piece cage system according to a second embodiment of the present invention.

FIG. 14 depicts an end member and a base member assembly of a multi-piece cage system according to the second embodiment of the present invention in a partially assembled state.

FIG. 15 depicts an end member and a base member assembly of a multi-piece cage system according to the second embodiment of the present invention in an assembled state.

FIG. 16 illustrates a top plan view of two supports used for a base member assembly of a cage system according to the second embodiment of the present invention.

FIG. 17 illustrates a front view of the supports of a base member assembly of a cage system according to the second embodiment inserted between two adjacent vertebrae and positioned using a lifting device.

FIG. 18 illustrates a side plan view of a link for use in a base member assembly of a cage according to the second embodiment of the present invention.

FIG. 19A shows a front plan view of a containment plate for use with a cage system according to the first embodiment of the present invention.

FIG. 19B illustrates a cross-sectional view of the containment plate of FIG. 19A, viewed in the direction of the arrows.

FIG. 20A illustrates a front plan view of a containment plate for use with a cage system according to the second embodiment of the present invention.

FIG. 20B illustrates a cross-sectional view of the containment plate of FIG. 20A, viewed in the direction of the arrows.

FIG. 21 illustrates an arrangement for coupling a wedge portion of a link to a sidewall of a support according to one variation of the present invention.

FIG. 22 illustrates a front plan view of one variation of an end member for a cage according to the present invention.

FIG. 23 illustrates a front plan view of another variation of an end member for a cage according to the present invention.

FIG. 24 illustrates a cage system with one of the end members illustrated in FIGS. 22 and 23 installed between adjacent vertebrae and secured to the adjacent vertebrae with screws.

### DETAILED DESCRIPTION

Referring to FIG. 1, a multi-piece cage system according to the first embodiment of the present invention includes a base member assembly 10. Base member assembly 10 includes two supports each including two generally parallel sidewalls 11, 12 and an end wall 14. In this embodiment, sidewalls 12 are shorter than sidewalls 11. Each sidewall 11, 12 includes a terminal, free end 16 and an end 18 opposite its free end 16. End wall 14 extends from one end 18 of one sidewall 12 to another end 18 of the other sidewall 12. Preferably, each support of base member assembly 10 is a generally elongated U-shaped body, although other shapes may be employed without deviating from the scope and spirit of the present invention.

In this embodiment, a link 13 is positioned at free ends of sidewalls 12 and couples sidewalls 12 to one another. Preferably, once link 13 is assembled in place, the length of sidewalls 11, and the total length of sidewalls 12 and link 13 will be equal.

A first elongated tie 19 is integrated with sidewalls 11 and extends longitudinally away from ends 16 thereof.

A second elongated tie 20 is integrated with link 13 and extends longitudinally away from link 13 preferably in a direction parallel to the direction of extension of tie 19.

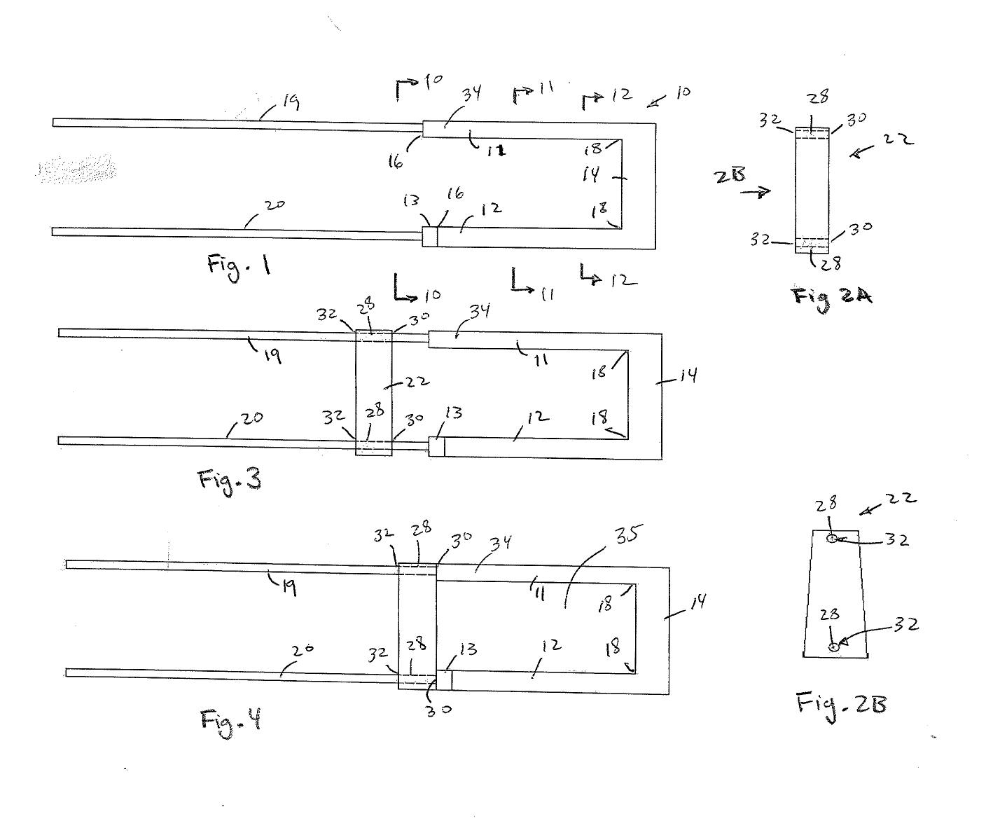

Ties 19, 20 may be metallic bodies (e.g. titanium rods). Ties 19, 20 function to align an end member 22 (see FIGS. 2A, 2B) to base member assembly 10 as further disclosed below.

Referring now to FIGS. 2A and 2B, an end member 22 for a multi-piece cage system according to the present invention includes features for receiving ties 19, 20. Thus, end member 22 may be a closure wall 24 that is at least wider than the distance between terminal, free ends 16, whereby, when end member 22 is integrated (i.e. mated with, coupled to, and/or locked with) with base member assembly 10, the two bodies form a body that surrounds a space for receiving material that promotes bone growth as further described below.

According to one aspect of the present invention, end member 22 includes two elongated channels 28 each defined to extend through its body. Each channel 28 includes a first opening 30 and a second opening 32 opposite first opening 30.

Referring now to FIG. 3, each tie 19, 20 is received inside a respective channel 28 at a respective first opening 30 and exits from the oppositely positioned second opening 32 as end member 22 is moved toward base member assembly 10, which, in practice, has been inserted in an intervertebral position between adjacent vertebrae. Thus, ties 19, 20 serve to properly register and align end member 22 with base member assembly 10.

As illustrated in FIG. 4, end member 22 is further guided along ties 19, 20 until end member 22 and base member assembly 10 mate with one another to realize a cage. Note that in an assembled state, end member 22 and base member assembly 10 define a space 35 that can retain material for promoting bone growth as is known in the field.

Each channel 28 may have an oval cross-section (transverse to its longitudinal axis) and may be sized to make contact with the exterior surface of a respective tie 19, 20, whereby ties 19, 20 and channels 28 slidably engage one another. Preferably, the cross-section (transverse to its longitudinal axis) of each tie 19,20 would have the same shape as the cross-section of a channel 28 in which it is receivable, however, such is not a necessity. It should be noted that a ties 19, 20 may have a cross-section other than oval. For example, circular, square, triangular, rectangular, star-shaped, or any other shape would be within the scope of the present invention.

At least a portion of each tie 19, 20 that would extend beyond the boundaries of end member 22 should be configured so that it may be readily severed once the cage is fully assembled.

It can be appreciated by a skilled person, that in addition to ties 19, 20, a cage system according to the present invention can include a locking mechanism to integrate end member 22 and base member 10.

A suitable material for making a cage assembly 10 according to the present invention may be PEEK (polyether ether ketone), titanium or a titanium alloy, or carbon fiber.

Ties 19, 20 may be made from titanium, tantalum, stainless steel, or any other suitable metal alloy. Preferably, a cage system according to the present invention is made of a combination of different materials. For example, base member assembly 10 and end member 22 may be made of PEEK, while ties 19, 20 may be made from a titanium alloy.

Ties 19, 20 may be from 80 mm to 180 mm long. The length of ties 19, 20 would depend on which side of the patient is to receive the cage system.

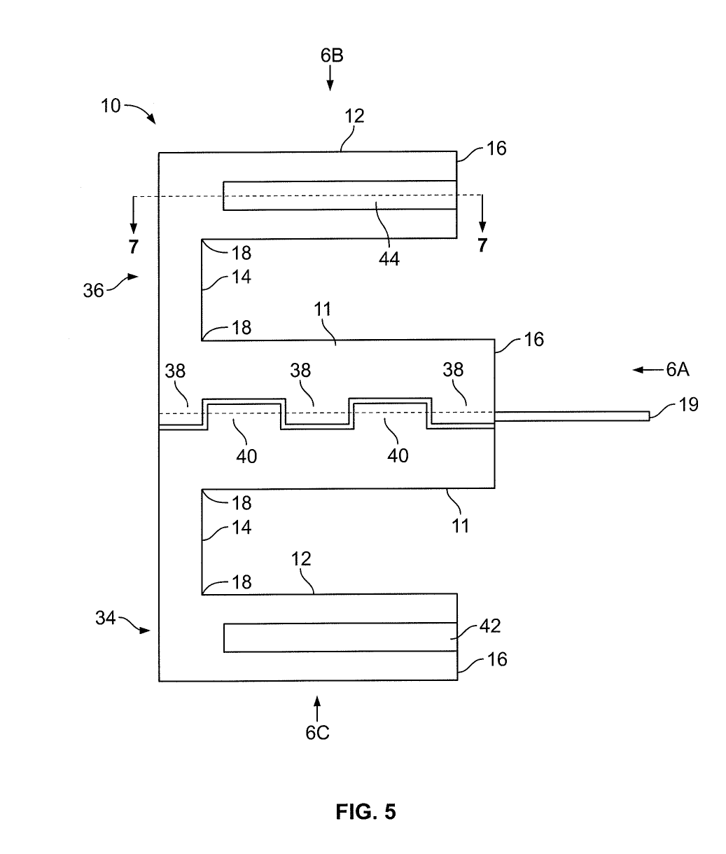

Referring now to FIG. 5, a base member assembly 10 in a first embodiment of the present invention includes a first support 34 and a second support 36. Each support is generally U-shaped and includes a long sidewall 11, a short sidewall 12 (shorter than long sidewall 11), and an end wall 14 extending between sidewalls 11, 12 as earlier described. Sidewalls 11, 12 preferably extend parallel to one another.

In the first embodiment, long sidewalls 11 of supports 34, 36 are coupled to one another to realize a hinge. Specifically, one end of elongated tie 19 is inserted and received in respective channels defined in spaced projections 38, 40 residing along sidewalls 11. As illustrated, three spaced projection 38 are provided along the length of sidewall 11 of support 36 and two spaced projections 40 are provided along the length of sidewall 11 of support 34. Projections 40 are received in spaces between projections 38. A channel defined in each projection 38, 40 registers (is aligned with) a channel of another, adjacently positioned projection 38,40. Tie 19 is received through channels of all projections 38, 40 and serves as a pivot or hinge pin allowing supports 34, 36 to rotate about the longitudinal axis of tie 19, whereby base member assembly 10 may be folded like a book.

Each sidewall 12 includes a groove 42, 44 defined therein. Each groove 42,44 extends to a free end 16, and extends preferably only along part of the length of a wall 12 and preferably terminates before reaching end wall 14. Further, preferably each projection 38,40 includes a convex exterior surface opposite a concave surface.

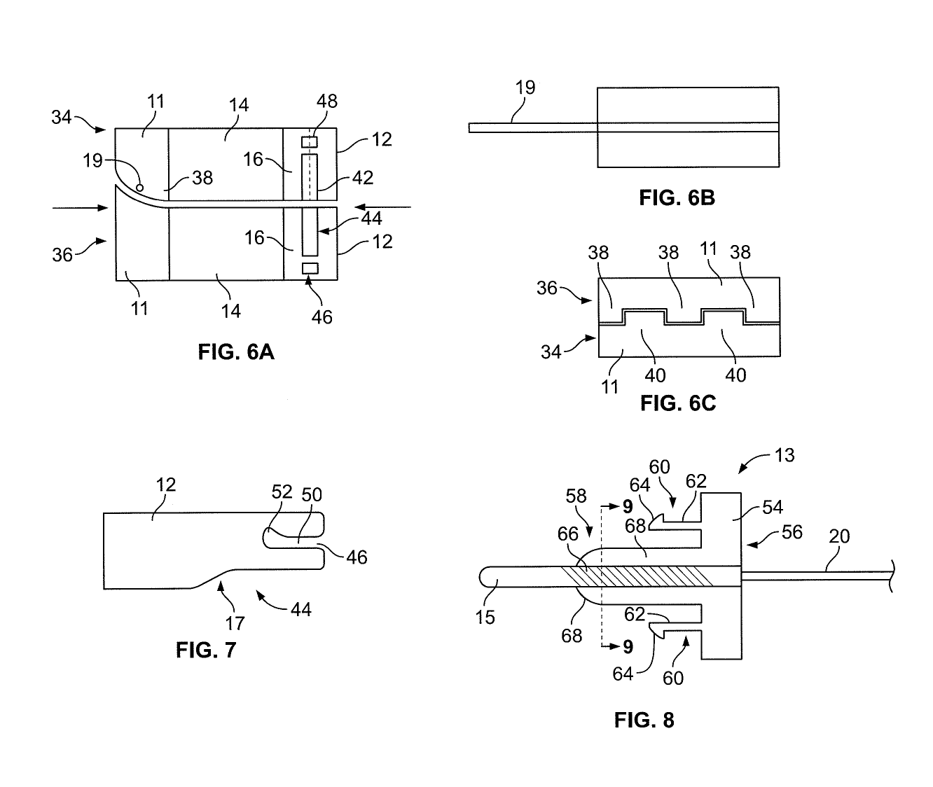

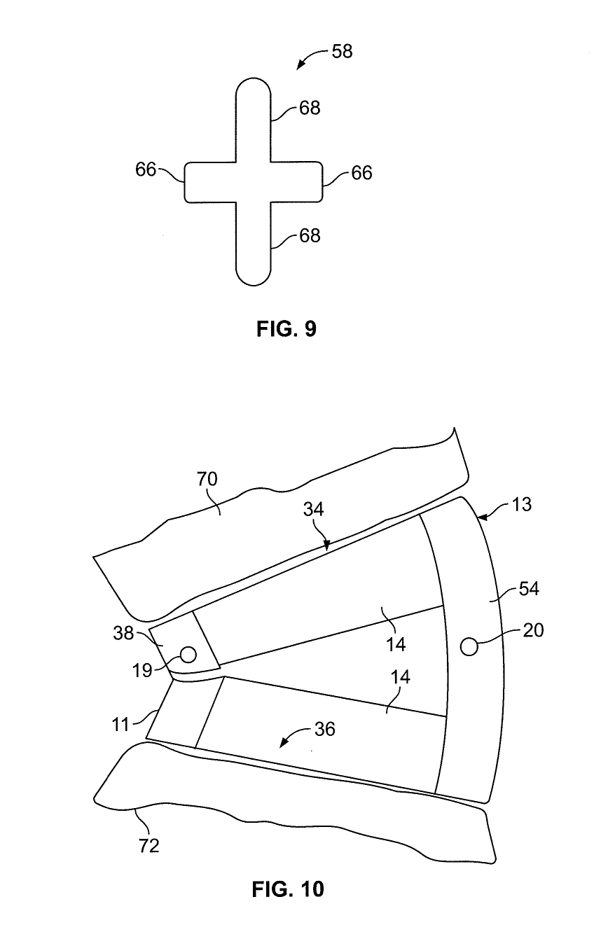

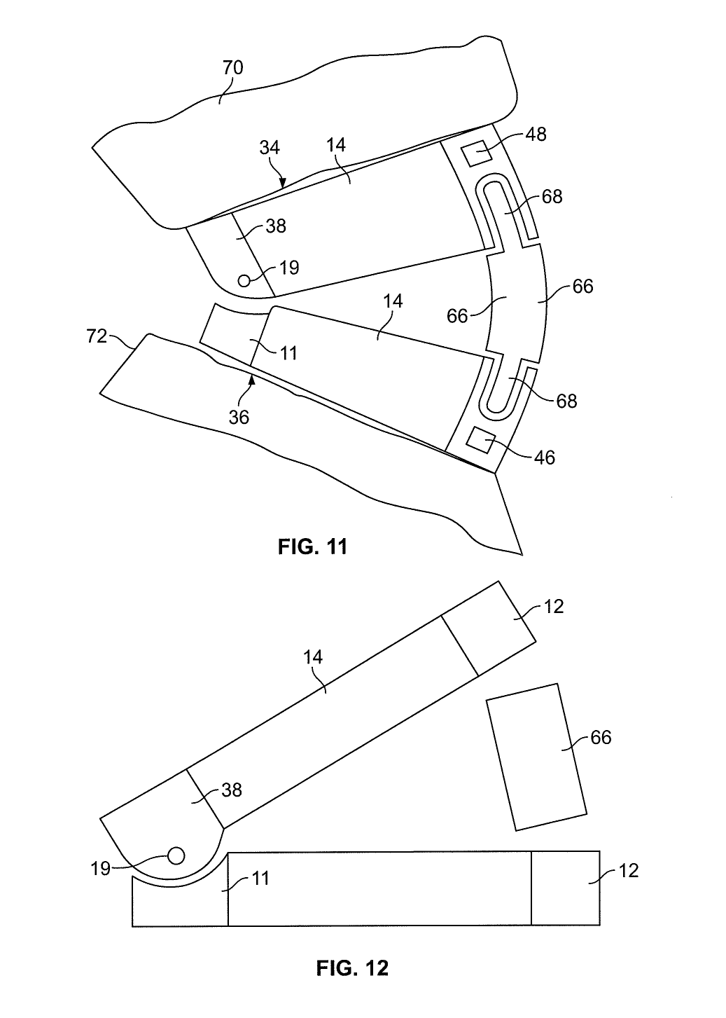

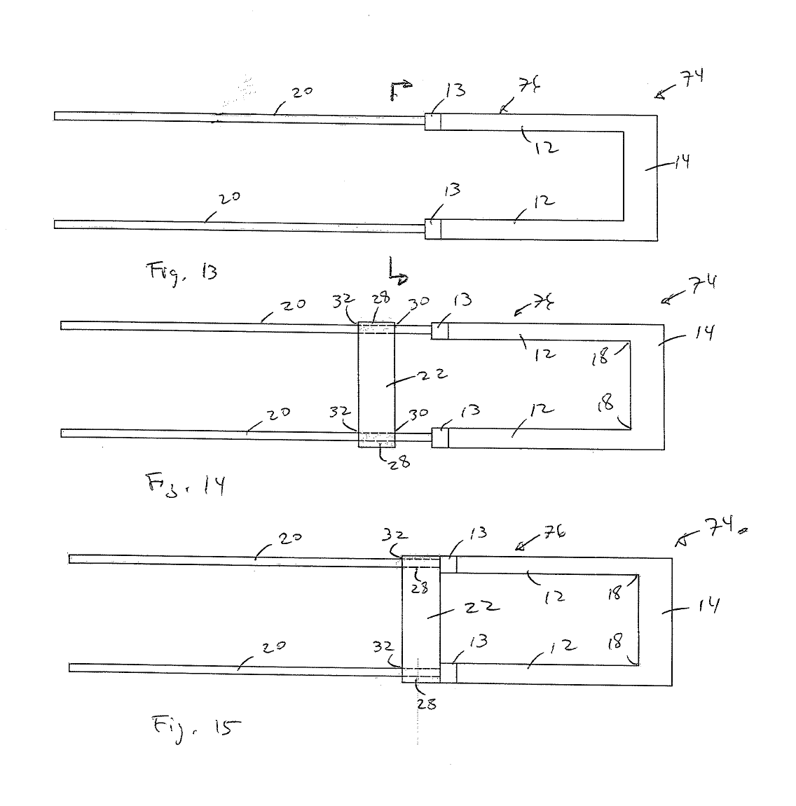

Referring now to FIG. 6, when base member assembly 10 is in a closed state, grooves 42, 44 face one another. Preferably, each sidewall 12 of supports 34, 36 further includes a receptacle 46, 48 defined therein and open at free end 16.

FIG. 7 illustrates a cross-sectional view along line 7-7 in FIG. 6, viewed in the direction of the arrows. As illustrated, receptacle 46 includes a narrow channel 50 extending to an enlarged end chamber 52. Receptacle 48 would have the same configuration.

Referring to FIG. 8, link 13 includes bridge section 54. Tie 20 is integrated with link 13 and extends away from a first surface 56 of bridge section 54. Extending in a direction opposite to the direction of extension of tie 20 are a shimmy section 58 and two elongated anchors 60 each positioned on a respective side of shimmy section 58. Each anchor 60 includes a neck 62 and an elongated free end 64. Each anchor 60 is dimensioned to be received in a respective receptacle 50. Once fully received in a receptacle 50, the enlarged free end 64 of an anchor 60 resides in enlarged end chamber 52 of the receptacle 50, whereby the retraction of link 13 is hindered because of the abutment of enlarged free end 64 and the interior walls of enlarged end chamber 52.

Referring to FIGS. 8 and 9, shimmy section 58 included two opposing wedge sections 66 which extend in a direction transverse to the direction of elongation of shimmy section 58. Shimmy section 58 further includes two guide lobes 68 oriented transverse to wedge sections 66. Each lobe 68 is dimensioned to be received in a groove 42, 44. Once lobes 68 are received in grooves 42, 44, wedges 66 are received between sidewalls 12. Thus, wedges 66 space sidewalls 12 of supports 34,36. Preferably, wedges 66 only extend as far as grooves 42, 44. When installing link 13, the leading edge 15 of link 13 enters grooves 42, 44 first. Then, the leading edge abuts ramp section 17 at the end of grooves 42, 44. While leading edge 15 is moved to the end of grooves 42, 44, ramp sections 17 of grooves 42, 44 force the dilation of supports 34, 36. It should be noted that lengths of leading edge 15, lobes 68 and wedges 66 may be different. Consequently, by selecting a link 13 with wedges 66 of different thicknesses the height (thickness) of base member assembly 10 can be changed in situ (i.e. during the operation).

FIG. 10 shows an end view of a base member assembly 10 with a link 13 (viewed in the direction of arrows 10-10 in FIG. 1) inserted between adjacent vertebrae 70,72. As shown, by selecting a properly sized link 13, the height of base member assembly 10 along walls 12 can be set so that first support 34 and second support 36 make intimate contact with adjacent vertebrae 70,72. FIG. 11 shows a cross-sectional view along line 11-11 (FIG. 1), viewed in the direction of the arrows and FIG. 12 shows a cross-sectional view along line 12-12 (FIG. 1) viewed in the direction of the arrows. Note that, preferably, lobes 68 and wedges 66 have the same thickness.

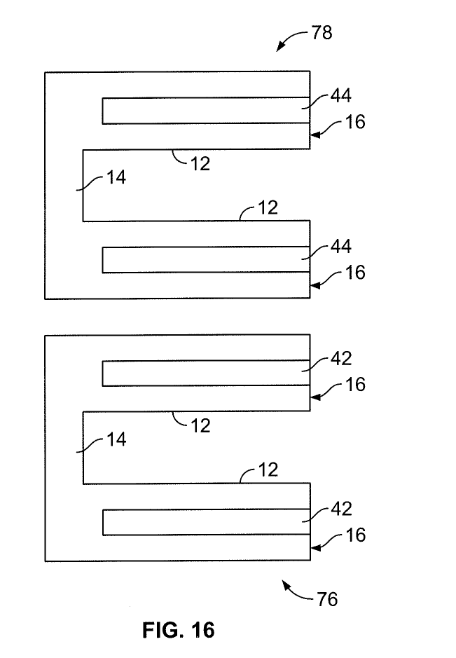

In a cage system according to the second embodiment of the present invention independent (unlinked) supports 76, 78 (see FIG. 16) are integrated with two links 13 to realize a base support assembly 74.

Referring to FIGS. 13, 14, 15, an end member 22 is assembled with base support assembly 74 to realize a cage in the same manner as the first embodiment. In this embodiment, however, each support 76 includes two sidewalls configured to have the features of sidewalls 12 of supports 34, 36.

FIG. 16 illustrates a plan view of supports 76, 79 in a disassembled state.

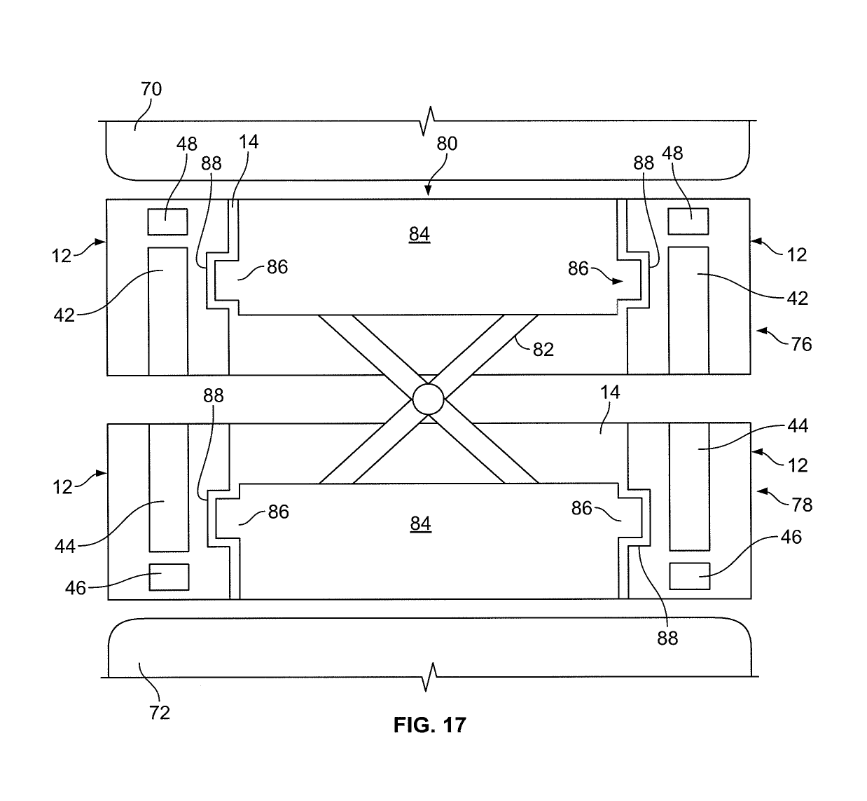

FIG. 17 illustrates a front plan view of supports 76, 78 positioned in a space defined between two adjacent vertebrae 70, 72 prior to integrating base member assembly 74. To position supports 76, 78 a lifting device (dilator) 80 may be employed. Lifting device 80 is configured to articulate and to engage 76, 78 in order to move supports 76,78 inside the space defined between adjacent vertebrae 70, 72 until supports 76, 78 make proper contact with vertebrae 70, 72.

Device 80 may include an expander 82 that can articulate and move two oppositely disposed engagers 84. Each engager 84 includes two oppositely positioned projections 86. Each projection 86 is received inside a groove 88 defined in an interior surface of a wall 12 to engage wall 12. Once engaged, engagers 84 move supports 76,78 relative to one another until supports 76,78 make intimate contact with adjacent vertebrae 70,72. Movement of engagers 84 is realized by articulating expander 82. Once supports 76, 78 are positioned, links 13 with proper wedge thicknesses are assembled onto supports 76, 78 to realize a base support assembly 74. Thereafter, device 80 is removed.

In both embodiments, once a base support assembly 10,74 is in place, end plate preparation is performed, graft material is deposited inside base support assembly 10, 74, and an end member 22 is installed to complete the cage.

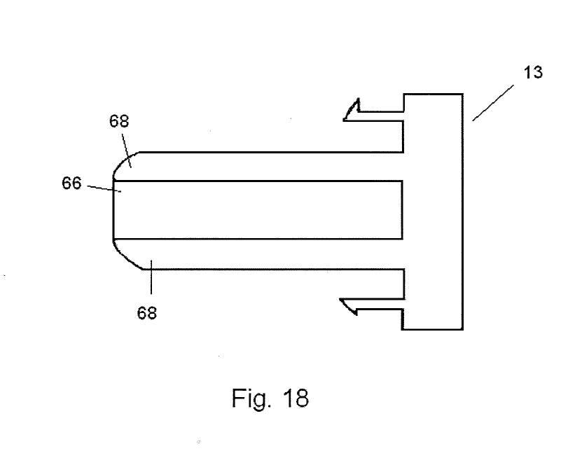

It should be noted that grooves 42, 44 in supports 76, 78 need not include ramp sections since dilation is performed by device 80. In this embodiment, link 13 would not include a leading edge 15. A link 13 is suitable for the embodiment as illustrated in FIG. 18.

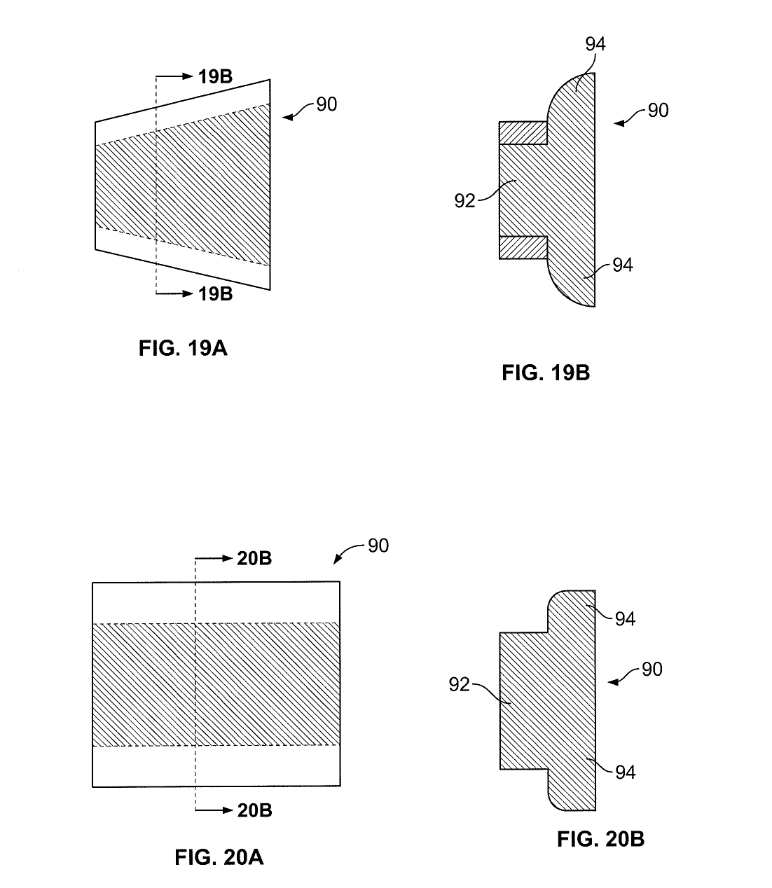

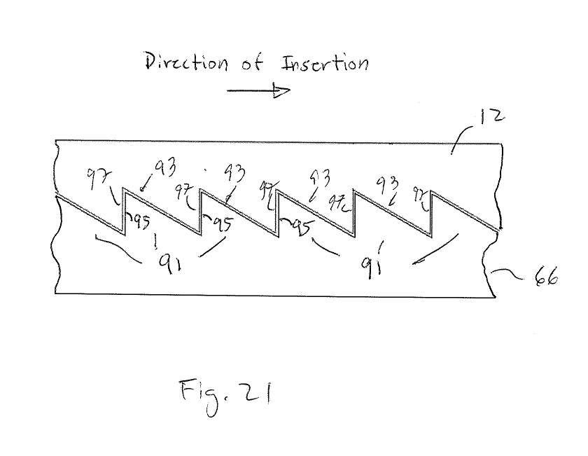

Note that to ensure that the graft material is contained inside the cage, a containment plate 90 (FIGS. 19A, 19B, 20A, 20B) may be inserted into base member assembly 10, 74 before deposition of the graft material. Containment plate 90 may be a flat plate sized to span (extend between) end walls 14 of the supports to cover the gap between the supports resulting from dilation and to contain the graft material within the cage. FIG. 19A shows a front plan view of a containment plate 90 for use with a system according to the first embodiment. FIG. 20A illustrates containment plate for use with a system according to the second embodiment. Note that in each case, containment plate includes an enlarged section 92 configured to fit inside the gap and flange sections 94 that extend beyond respective edges of the gap.

Referring to FIG. 21, to further strengthen the coupling between link 13 and supports 34,36/76,78, a plurality of notches may be defined on the surfaces of wedges 66 that meet with surfaces of walls 12 adjacent grooves 42, 44. The notches so defined result in a plurality of spaced teeth 91, which are receivable in corresponding notches 93 defined on surfaces of sidewalls 12 adjacent grooves 42, 44. Each tooth 91 is preferably provided with an abutment wall 95, which is oriented preferably 90° relative to the direction of insertion of link 13 into grooves 42, 44. Each abutment wall 95 abuts an abutment wall 97 inside a notch 93, which is preferably oriented 90° relative to the direction of insertion of link 13. Thus, once wedges 66 are received between sidewalls 12, abutment walls 95, 97 hinder the withdrawal of link 13. The configuration illustrated in FIG. 21 may be applied to links in both embodiments and to one or more wedges thereof.

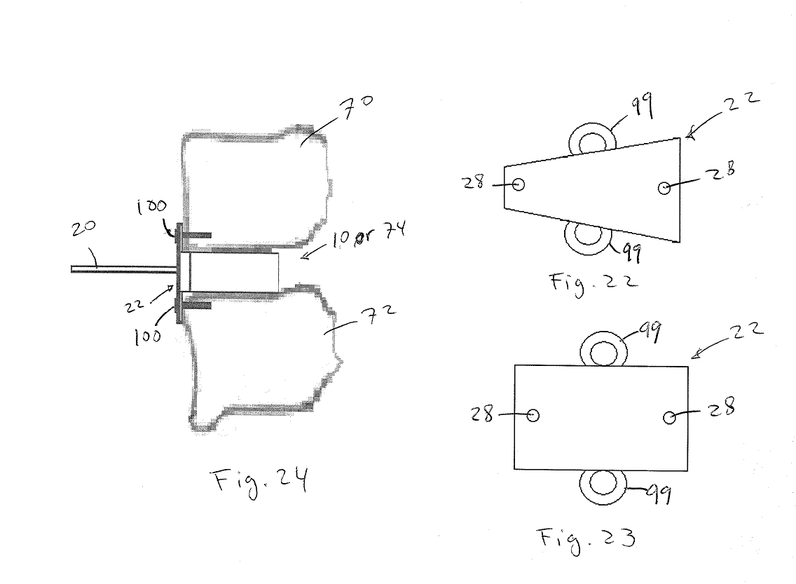

Referring to FIG. 22, an end member 22 may include two eyelets 99 extending away from opposing edges thereof. Each eyelet 99 may be sized to receive a screw 100 whereby end member 22 may be secured to the adjacent vertebrae 70,72 thereby further securing the cage in place once the cage is installed between adjacent vertebrae 70,72 as illustrated in FIG. 24.

FIG. 23 illustrates another variation of an end member 22 that includes eyelets 99 for receiving screws 100. It should be noted that each eyelet 99 extends in a direction that crosses (preferably at a 90 degree angle) to the plane that intersects channels 28 in end member 22.

While the cage systems disclosed herein are particularly suited for lateral applications, the cage systems may also be employed in anterior applications.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A system for intervetebral fusion comprising:
an intervetebral cage that includes a base member and an end member, the base member including a first support and a second support, the first support and the second support each including a first sidewall, a second sidewall spaced from the first sidewall, and an end wall connecting the first sidewall to the second sidewall, the end member being configured for assembly with the base member to define a space to receive bone graft material.

2. The system of claim 1, wherein the sidewalls are generally parallel to one another.

3. The system of claim 1, wherein the end member is configured for assembly to ends of the sidewalls of the first and the second support members.

4. The system of claim 1, wherein the first sidewalls of the first and the second support members are hingedly coupled to one another, and the second sidewalls may be displaced relative to one another.

5. The system of claim 4, further comprising a link that couples the second sidewalls of the first and the second support members.

6. The system of claim 1, further comprising a first tie and a second tie, wherein the end member is assembled onto the base member with the first tie and the second tie.

7. The system of claim 1, further comprising a link that couples the first sidewalls or the second sidewalls of the first support and the second support.

8. The system of claim 7, wherein the link includes a wedge or a plurality of wedges configured to be received between the first sidewalls or the second sidewalls.

9. The system of claim 8, wherein the link includes an anchor that is received by a first sidewall or a second sidewall.

10. The system of claim 9, wherein the link includes a lobe that is received in a groove defined in a first sidewall or a second sidewall.

11. The system of claim 8, wherein the link includes a lobe that is received in a groove defined in a first sidewall or a second sidewall.

12. The system of claim 1, further comprising a first tie, wherein the first sidewalls of the first and the second support members are coupled to rotate about the first tie, a link that couples the second sidewalls of the first and the second supports, and a second tie extending from the link, wherein the end member is configured to receive the first tie and the second tie.

13. The system of claim 1, further comprising a first link and a second link, the first link being configured to couple the first sidewalls of the first and the second supports and the second link being configured to couple the second sidewalls of the first and the second supports.

14. The system of claim 13, further comprising a dilator.

15. The system of claim 1, further comprising a first tie and a second tie, wherein the end member includes a first opening that receives the first tie and a second opening that receives the second tie, wherein the first tie and the second tie are coupled to the base member, and wherein the end member includes a plurality of apertures each for receiving a screw.

16. The system of claim 1, further comprising a containment plate configured to be received in a spaced defined by the base member to close a gap between the first support and the second support.

17. The system of claim 1, further comprising a link that is configured to couple the first sidewalls or the second sidewalls, the link including a wedge that is received between the first sidewalls or the second sidewalls, the wedge including notches that are received in respective recesses defined in the first sidewalls or the second sidewalls.

18. The system of claim 1, further comprising a link that couples the first sidewalls or the second sidewalls, the link including two lobes each received in a respective groove defined in a respective sidewall and two wedges each being disposable between the first sidewalls or the second sidewalls on a respective side of a respective groove, the link further including two anchors each being receivable in a respective receptacle defined in a respective sidewall.
